(19) United States
(12) Patent Application Publication
(10) Pub. No.: US 2024/0350297 A1
(43) Pub. Date: Oct. 24, 2024

(54) Condomless External Catheter

(71) Applicants: Zachary Walter Newman, Van Nuys, CA (US); Abbie Riva Harooni, Van Nuys, CA (US)

(72) Inventors: Zachary Walter Newman, Van Nuys, CA (US); Abbie Riva Harooni, Van Nuys, CA (US)

(21) Appl. No.: 18/640,918

(22) Filed: Apr. 19, 2024

Related U.S. Application Data
(63) Continuation-in-part of application No. 18/445,574, filed on Oct. 25, 2023.
(60) Provisional application No. 63/577,336, filed on Apr. 19, 2023.

(51) Int. Cl. A61F 5/453; A61F 5/44; A61F 5/441

(52) U.S. Cl. CPC A61F 5/453; A61F 5/4401; A61F 5/4407; A61F 5/441

(57) ABSTRACT

A urine collection system for a male person generally includes a portable collection pouch having a chamber configured to receiving an absorbent pad. A penis receptable having an elastomeric band securely positions the penis of the person so that urine enters the chamber. Pull tabs can be used to expand an opening defined by the band. The absorbent pad has a hole or is otherwise configured such that the penis shaft can extend through the pad. An opposite side of the pouch from the penis receptable can have an opening through which a Foley catheter can be inserted. The pouch can have another opening for insertion and removal of pads.

### PRIORITY CLAIM

This application is a Continuation-In-Part of U.S. non-provisional application Ser. No. 18/445,574, filed Nov. 24, 2023, which claims priority to U.S. provisional application Ser. No. 63/577,336 filed Apr. 19, 2023 and U.S. provisional application Ser. No. 63/576,774 and Mar. 9, 2023, each of which is incorporated herein by reference in their entirety. Where a definition or use of a term in a reference that is incorporated by reference is inconsistent or contrary to the definition of that term provided herein, the definition or use of that term provided herein is deemed to be controlling.

### FIELD OF THE INVENTION

The present disclosure relates generally to systems, devices, and methods for collecting and transporting urine away from a human.

### BACKGROUND

In various circumstances, a man may have limited or impaired mobility such that typical urination processes are challenging or impossible. For example, a person may experience or have a disability that impairs mobility. A person may have restricted travel conditions such as those experienced by pilots, drivers, and workers in hazardous areas. Additionally, urine collection is needed for monitoring purposes or clinical testing.

Urinary catheters, such as a Foley catheter, can be used to address some of these circumstances, such as incontinence. Unfortunately, however, urinary catheters can be uncomfortable, painful, and can lead to complications, such as infections. Prolonged use of Foley catheters can lead to a slit type of rip in the penis where the foley catheter starts leaking heavily, and can no longer capture all the discharged urine leaving the patient in continuously soiled condition.

Additionally, bed pans, which are receptacles used for the toileting of bedridden patients, such as those in a health care facility, are sometimes used. Bed pans, however, can be prone to discomfort, spills, and other hygiene issues. There are other external urinary catheters that can address these issues but they are also problematic. The most common type of external urinary catheter used is a condom type catheter which frequently falls off, creates lacerations on the penis making sexual intercourse painful, does not accommodate an erection, is inconvenient to use because it has to be disconnected prior to trips to bathroom at night for defecation, and finally can cause urinary tract infections similar to Foley indwelling catheters.

The second most common type of external catheter that do not use condoms are still very inconvenient to use because they require a clean shaven surface area on the pubic region of the male in order to adhere properly because they use an adhesive membrane for attachment. Furthermore the adhesive is prone to detachment during night time sleeping and the system still relies on a tube and a collection bag at the side of the bed in order to function. This also creates the same problem as the condom type catheter where it has to be disconnected during night-trips to bathroom for the purposes of defection.

The third most popular external catheter requires a vacuum and suction and can only be used in hospital setting where a vacuum pump is readily available. There are other contraptions on the market, but none offer a satisfactory solution. Contraptions that are bizarre or unsightly in design, can produce a negative body image. Thus, there is a need for a device capable of collecting urine from a man comfortably and with minimal contamination of the user and the surrounding area while solving the all the problems associated with the use of aforementioned Foley indwelling and external catheters all the while being comfortable to use, portable, self-contained (not requiring attachments,) as well as clandestine under use.

Many prior art catheter designs fail during back lying (supine), side lying (syms) positions, during erections and retractions. The improvements herein allows erections, allow use during sleep, prevents retractions, all the while keeping the catheter in place, functioning properly. Alternative prior art designs require a vacuum pump, are not self-contained, are only available at hospital settings, are not portable, and further require use of adhesives to secure the catheter to the body of the patient. As such, if the patient's body parts are not clean shaven or the adhesives fails, the whole system will fail. The use of adhesives may also irritate the skin over time. Adhesive securing prior art catheters are often subject to failure after a few days of hair growth underneath the contact area.

There are hundreds of thousands of males with spinal cord injuries, many paraplegics, and quadriplegics. Most paraplegics and quadriplegics routinely rely on a caretaker for diaper changes and catheter placement and emptying. Many will wait hours in urine soiled diapers and clothes before the caretaker arrives to help.

Thus, there is still a need for a self-contained, portable urinary collection device for male users that is reliable over many days of use, while avoiding damage to the skin of the user.

### SUMMARY OF THE INVENTION

The inventive subject matter provides a system, apparatus, and methods in which urine is collected in a pouch containing an absorbent material, and worn substantially about the pelvis.

In some embodiments the penis of the user is inserted directly into an opening in the pouch, and in other embodiments the penis of the user is inserted into a tube the delivers urine into the pouch. In either case there is an elastomeric band which constricts about the penis in position. The elastomeric band expands or contracts in correspondence with a change in dimension of the penis, thereby preventing leaking of urine. Positioning of the band about the penis can be facilitated by at least two pull tabs.

Bands are preferably sized and dimensions contact no more than 5 percent of the surface area of the penis at its smallest size.

In some embodiments the pouch has a sealable opening opposite the side facing the user (rear side) for insertion of a Foley catheter.

Pouches can be sealably waterproof, in many instances using one or more waterproof polymers. Pouches preferably comprise 90 percent or other significant amount of biodegradable material. Pouches are also at least partially transparent so that the user or caretaker can visualize the amount and color the urine being collected, as well as any diagnostic strips within the collection chamber.

In preferred embodiments pouches house an absorbent pad. Pads can be impregnated or coated with a deodorant, which can be in crystal or any other suitable form. Pads can also be impregnated or coated with an antiseptic. The deodorant and antiseptic can advantageously be located or at least concentrated towards edges of the pads, so that additional antiseptic is released as the amount of urine increases. The antiseptic can also be present in crystal or other suitable form.

Pouches can have a sealable opening for insertion and replacement of the pads. The pad insertion opening can advantageously be located at an end of the pouch. All suitable closures are contemplated, including a semi-airtight zipper and or a flap type closure. Collection chambers within the pouches can have any suitable number of pads.

Pouches are preferably gusseted to allow for expansion of the urine collection chamber as the pads expand with absorption of fluid. A key improvement of the invention over the prior art is that it can be used as a diaper at home, on the road, as well as healthcare setting due to its portability and self-containment. The urine collecting device is suitable for bedbound patients as well as Paraplegics, and quadriplegics because it uses capillary action of the absorbent material to collect the urine while the patient is at fowlers, supine, prone, or simms positions.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 is a transparent frontal view of a urine collection device.

FIG. 2 is a transparent rear view of the urine collection device of FIG. 1, showing insertion of an absorbent pad.

FIG. 3 is a lateral cross-sectional view of the pouch of FIG. 1.

FIG. 4 is another transparent rear view of the pouch of FIG. 1, showing alignment of the front and back openings in the pouch.

FIG. 5 is another transparent rear view of the pouch of FIG. 1 a plug that mates with the back opening in the pouch.

FIG. 6 shows an outline of a sagittal cross-section of a pelvic region of a male patient in supine position using an embodiment of the urine collection device.

FIG. 7 shows the outline of the sagittal cross-section of the pelvic region of the male patient of FIG. 6, with a Foley catheter inserted in the penis through a rear opening in the pouch.

### DETAILED DESCRIPTION

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

The recitation of ranges of values herein is merely intended to serve as a shorthand method of referring individually to each separate value falling within the range. Unless otherwise indicated herein, each individual value is incorporated into the specification as if it were individually recited herein, and ranges include their endpoints.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention. Unless a contrary meaning is explicitly stated, all ranges are inclusive of their endpoints, and open-ended ranges are to be interpreted as bounded on the open end by commercially feasible embodiments.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

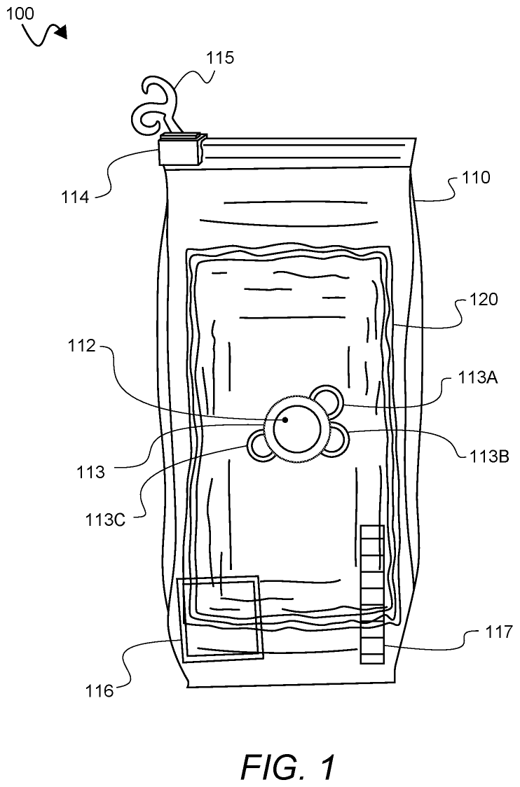

FIG. 1 is a front view of FIG. 1 (urine collection device 100), generally comprising a gusseted pouch 110 having opening 112, and closure 114. Pouch 110 houses absorbent pad 120.

Pouch 110 is preferably thin plastic, which when closed by closure 114 is substantially watertight. Contemplated pouches 110 can be any suitable size, including for example large medium and small sizes to accommodate large, medium, and small users. An exemplary small size of pouch 110 can be 4 inches by 6 inches, designed to collect urine for 5 hours for patients that are only suffering from mild dripping type of urine discharge. An exemplary medium size of pouch 110 can be 6 inches by 9 inches, designed collect urine for up to 9 hours on a low discharge level, and up to 4 hours for a normal urinary output level of 80 milliliters per hour. An exemplary large size of pouch 110 can be 9 inches by 11 inches, designed to replace an adult diaper, and can collect up to 12 hours of urine at normal human urinary output level of 80 ml/hr.

Examples of suitable polymers for the walls of pouch 110 are nylons, polyesters, polycarbonates, polyacrylates, polymers of ethylene-vinyl acetates and other acyl substituted cellulose acetates, non-degradable polyurethanes, polystyrenes, polyvinyl chloride, polyvinyl fluoride, poly (vinyl imidazole), chlorosulphonate polyolefins, polyethylene oxide, polyethylene terephthalate (PET), polytetrafluoroethylene (PTFE), and/or blends and copolymers thereof.

Pouch 110 is gusseted to allow for expansion as urine is taken up by pad 120. Pouch 120 is shown as substantially cuboid, although other shapes are also contemplated.

Absorbent pad 120 housed by pouch 110 is sized to accommodate the amount of urine to be collected, i.e., a small pad in a smaller pouch and a large pad in a larger pouch. Additionally, or alternatively, pouch 110 can concurrently house multiple absorbent pads 120.

Front opening 112 is bounded by an elastomeric band 113, that expands to receive the penis (not shown) of the user (not shown). Once the penis 640 is inserted, elastomeric band 113 contracts to form a snug fit around the penis.

Elastomeric band 113 can advantageously be attached to the pouch 110 via heat sealing, thereby creating a substantially leak proof seal despite changes in diameter of the penis. Elastomeric band 113 can alternatively be distanced from the pouch 110 by up to 2 cm or more. For longer distances a tube (not shown) can be used to provide a flow path from the penis 640 to the chamber (lumen) of the pouch 110.

Elastomeric band 113 preferably contacts at most 5% of a surface area of the penis. Different size bands can be used for different size users, where band widths and can advantageously range from 0.5 cm to 2 cm.

Elastomeric band 113 should not be confused with just a simple hole in order to insert the penis. First, elastomeric band 113 prevents backflow of urine out of the pouch; hence the urine collection device 100 can be used in a supine, prone, or syms positions while the user is awake or asleep. Secondly elastomeric band 113 aids to hold the pouch in place, again by its capability to contract to form-fit around the penis. Third, elastomeric band 113 expands to accommodate an erection, and subsequently spontaneously contracts to reform the snug fit once the erection has subsided. Fourth, elastomeric band 113 holds the penis in place, and does not allow it to retract.

In contrast to prior art devices, urine collection device 100 does not require adhesives, vacuum pumps, or shaved areas around the genitals to function properly. Moreover, urine collection device 100 is entirely portable, and thus can be used by ambulatory patients as well as the general public.

Still further, elastomeric band 113 has minimal surface contact with the penis, which minimizes possible irritation to the skin of the penis. Other designs such as the condom catheter. bag-on-penis catheter, or other designs using a sheath over the penis, have the potential to irritate the skin of the penis to the point of lacerations. In some embodiments elastomeric band 113 is only in contact with 3%-7% of a shrunken penis skin surface area, which contrasts with other sheath and non-sheath type catheters may cover anywhere from 20% to 100% percent of a shrunken penis surface area. The more surface area contact with the penis, the greater the chance of lacerations. An intact penis surface skin is a crucial benefit for sexually active paraplegics, quadriplegics, and others. No known designs address this very important issue. Elastomeric band 113 may be available in three or more different sizes in order to accommodate different size penises.

Elastomeric band 113 has pull tabs 113A, B and C attached to it which allow the band 113 to open and contract. Pull tabs 113A, B and C can directly attach to band 113, substantially as a small point of contact, or can be coupled to the band by semi-circular connectors, which would serve to more evenly expand the band 113. Pull tabs 113A, B and C are shown as rings so that a person operating the tabs can put a finger into a hole defined by the ring. However, pull tabs can have any suitable shape, which might or might be ring-shaped and might or might not have holes. Pull tabs 113A, B and C are substantially flat so that they lie unobtrusively against the front surface of the pouch 110.

Closure 114 is shown as a zipper type sliding device that mates front and back sides of the pouch 110 to provide a substantially water-tight seal. Handle 115 of the zipper has a large bidirectional “S” shaped handle suitable for paraplegics and quadriplegics with limited use of their upper digits to couple the pouch 110 to a belt or strap.

The anterior or posterior face of pouch 110 is furnished with a weight-to-urinary output conversion chart 116, which is used to measure the urinary output of patients. This simple system contrasts well with prior art that inaccurately estimates urinary output by determining the number of diaper changes per shift.

The diagnostic capabilities of the urine collection device 100 is further enhanced by the incorporation of urinary diagnostic strip 117 inside pouch 110. In contemplated embodiments, diagnostic strip 117 has the capability to alert the health care provider to conditions such as proteinuria, dehydration, and possible urinary tract infections.

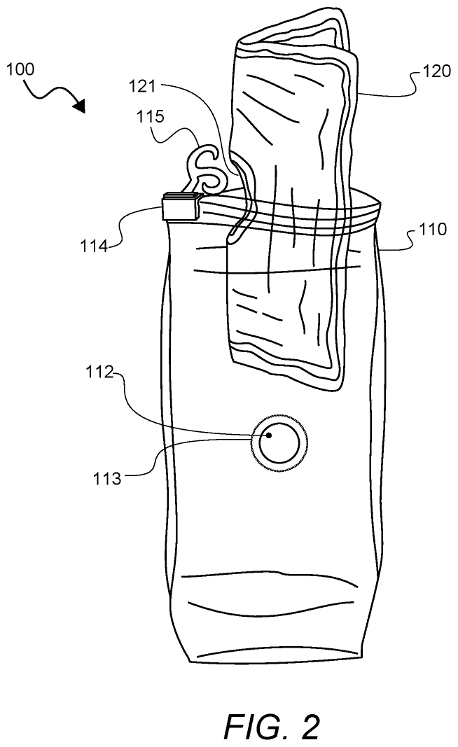

FIG. 2 is a transparent rear view of the urine collection device 110, showing insertion of an absorbent pad 120 into pouch 110. In contemplated embodiments the absorbent pad comprises an absorbent material, woven or non-woven, sized and dimensioned to fit inside pouch 110. The absorbent pad 120 has a hole 121 in the middle to accommodate the penis (not shown) of a user (not shown). Absorbent pad 120 is depicted as folded for insertion into pouch 110. Upon insertion pad 120 is unfolded and positioned so that hole 121 lines up with opening 112 in the pouch 110.

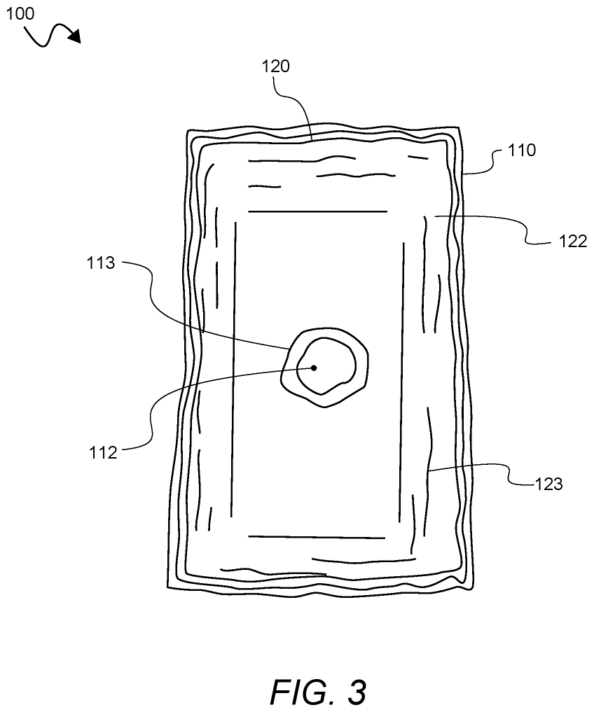

FIG. 3 is a lateral cross-sectional view of the pouch 110 of FIG. 1. Lateral regions of pad 120 are optionally impregnated or coated with a body-safe deodorant composition 122 to mitigate urine odor, and an antiseptic composition 123 to disinfect patients with a history of urinary tract infections. Each of the deodorant composition 122 and the antiseptic composition can be present in crystal or other suitable form.

By virtue of the deodorant composition 122 and antiseptic composition 123 being impregnated or coated in the lateral regions of pad 120 (which can include a laterally concentrated gradient), those compositions would not contact the penis until a urination event. This is an extra safety precaution. It only after a urination event that the crystals will dissolve and sanitize the discharged urine. The amount of antiseptic crystals can be adjusted in such way as to create the right aqueous concentration of the antiseptic in order to be body safe. Such disinfectants may be chlorohexidine crystals or sodium hypochlorite microencapsulation. Safer and more user friendly antiseptics can always be substituted.

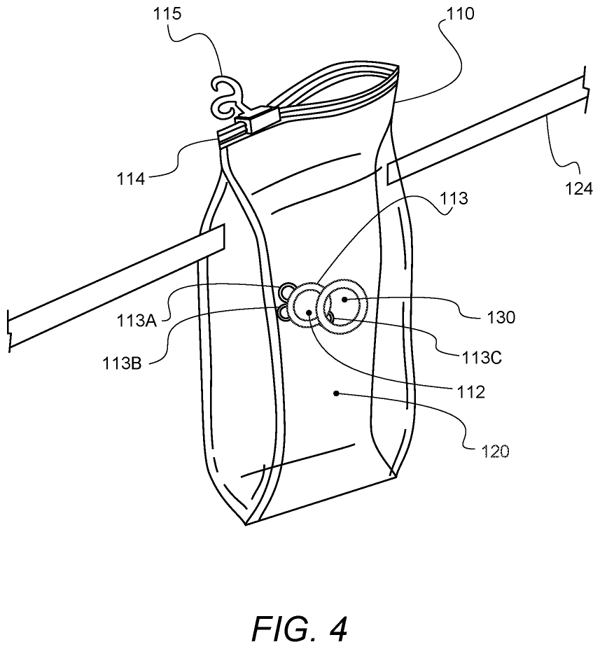

FIG. 4 is another transparent rear view of pouch 110, showing alignment of front 112 and rear 130 openings in pouch 110. Strap 124 is attached to sides of pouch 110, and is sufficiently long to encircle the waist of the user.

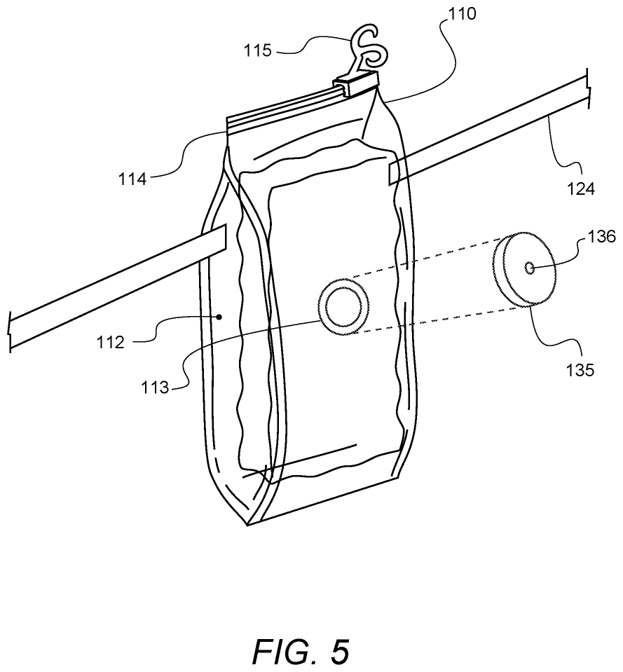

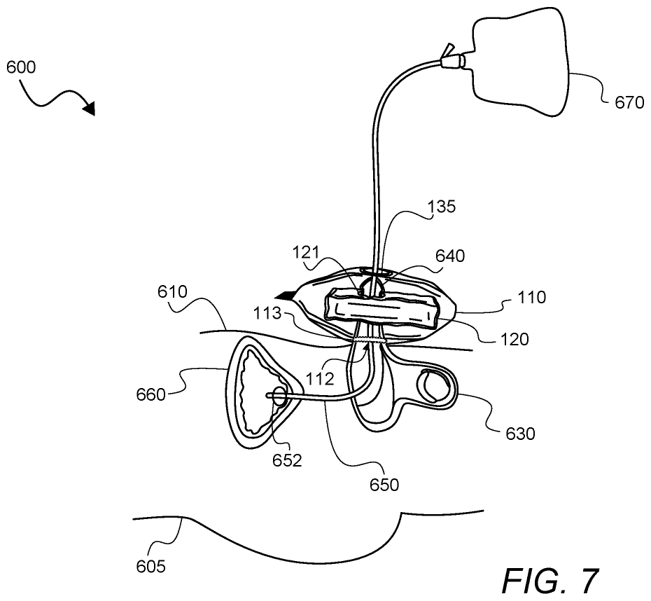

FIG. 5 is another transparent rear view of pouch 110, showing an exploded view of a plug 135 that mates with the back opening in the pouch 130. Plug 135 includes a self-sealing hole 136 sized to accommodate a Foley catheter (see FIG. 7). Plug 135 is optionally removable from pouch 110 to allow rapid draining of any urine from pouch 110 that is not absorbed by pad 120.

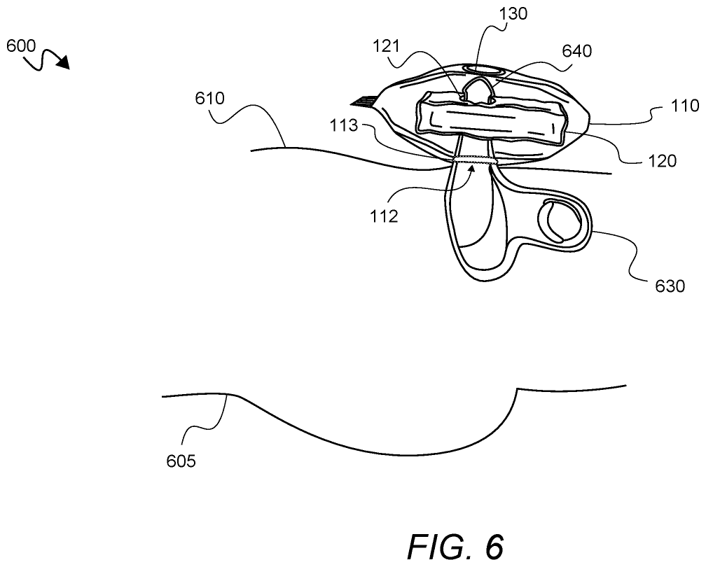

FIG. 6 shows an outline of a sagittal cross section of a pelvic region 610 of a male patient 605 in a supine position, using an embodiment of the urine collection device. The testes 630 of patient 605 face the front side of pouch 110. The penis 640 of the patient 605 passes 640 through elastomeric band 113, through front opening 112 in pouch 110, through hole 121 in pad 120, and approaches or abuts rear opening 130 in pouch 110.

FIG. 7 shows the outline of the sagittal cross-section of the pelvic region 610 of the male patient 605 of FIG. 6. Foley catheter 650 passes through the self-sealing hole 136 of plug 135, through the shaft of penis 640, and along with balloon 652 into bladder 660. At its other end Foley catheter 650 drains into urine collection bag 670, containing any discharged urine that leaks out of the absorbent pad 120

The embodiment of FIG. 7 is specifically designed for patients who have suffered from urinary retention for many years and have been using Foley type catheters for years. Foley type catheters, with long term use, start leaking heavily. The leakage eventually gets to a point that the catheter can only remove ½ of the urine discharge. The other half of the urine discharge simply leaks all over the patient's bed or clothing. A diaper cannot be used in conjunction with the Foley catheter because the Foley catheter is in the way. In this configuration, any urine that is not collected by the Foley, will be collected by the disclosed urine collection device 100, leaving the patient clean, dry, comfortable, free from skin irritation and pressure sores.

### BENEFITS

Among the many benefits of the present our catheter design is that many of the parts are reusable. Of course, pads 120 can be replaced, but all of the other components, including pouches, can be reused. In contrast, most known catheters such as the condom catheter, bag over penis catheter, other sheath type catheters, as well as the vacuum assisted urinary catheter, contain parts that are not reusable. In addition, interior pads can be biodegradable.

Another benefit is that embodiments of the device can be used for long periods of time, for example all night or all day, even when the user is sleeping. This feature is important for a number of reasons. First it allows for the timely repositioning of the bedbound patient during day or night. Most doctors' orders require the repositioning of the bedbound patients every 2 hours in order to prevent bedsores. Secondly, nursing homes are often understaffed and patients have to wait for hours for a diaper change.

Still another benefit is that embodiments of the device can keep unbounded urine away from the surface of the skin for up to 12 hours, or perhaps even longer. This feature will greatly reduce the number of skin breakdowns associated prolonged urine-skin contact. It also has the added benefit of freeing the hands of personnel performing diaper changes, in order to perform less diaper changes and more patient repositioning. This arrangement ultimately will greatly reduce the number of bedsores in facilities and the subsequent wound care needs associated with it. Bed sore wounds are anywhere from unsightly and malodorous to downright painful at the beginning of their formation, not to mention that their treatment costs taxpayers multi-millions of dollars per year. Every healthcare facility has 2-4 nurses specifically dedicated to treating pressure sore wounds.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context.

What is claimed is:

1. A urine collection device for use with a penis of a user, comprising:
a portable collection pouch having a chamber configured to receive an absorbent pad;
a penis receptacle having an elastomeric band and at least two pull tabs coupled to the band such that pulling on the pull tabs expands an opening defined by the band; and
the receptacle positioned such that urine from the penis flows to the chamber.

2. The device of claim 1, wherein the receptable is within 2 cm of an opening in the pouch.

3. The device of claim 1, wherein the elastomeric band is at most 1 cm wide.

4. The device of claim 1, wherein the pad includes a passageway through which the penis can extend.

5. The device of claim 4, wherein the passageway comprises a hold in the pad.

6. The device of claim 1, wherein the pad includes at least one of a deodorant crystal and a deodorant compound concentrated in a lateral region of the pad.

7. The device of claim 1, wherein the pad includes at least one of an antiseptic crystal and an antiseptic compound concentrated in a lateral region of the pad.

8. The device of claim 1, further comprising a one of a semi-airtight zipper or a flap type closure sized and dimensioned to allow insertion of the pad.

9. The device of claim 1, wherein the pouch further comprises an opening configured to receive a plug, and the plug having an opening configured to receive a portion of a catheter from outside of the pouch.

10. A urine collection device for use with a penis of a user, comprising:
a portable collection pouch having a chamber configured to receive an absorbent pad;
a penis receptacle positioned such that urine from the penis flows to the chamber; and
wherein the pad includes a passageway through which the penis can extend.

11. The device of claim 10, wherein the penis receptacle has an elastomeric band and at least two pull tabs coupled to the band such that pulling on the pull tabs expands an opening defined by the band.

12. The device of claim 1, further comprising an absorbent pad disposed within the pouch, wherein one of a deodorant crystal or a deodorant compound is disposed or at least concentrated at edges of the pad.

13. The device of claim 1, wherein the pad includes at least one of a deodorant crystal and a deodorant compound concentrated in a lateral region of the pad.

14. The device of claim 1, wherein the pad includes at least one of an antiseptic crystal and an antiseptic compound concentrated in a lateral region of the pad.

15. The device of claim 1, further comprising a one of a semi-airtight zipper or a flap type closure sized and dimensioned to allow insertion of the pad.

16. The device of claim 1, wherein the pouch further comprises an opening configured to receive a plug, and the plug having an opening configured to receive a portion of a catheter from outside of the pouch.
